(19) United States
(12) Patent Application Publication
(10) Pub. No.: US 2022/0257145 A1
(43) Pub. Date: Aug. 18, 2022

(54) SYSTEMS AND METHODS FOR COMPUTER ASSISTED FEMORAL SURGERY

(71) Applicant: INTELLIJOINT SURGICAL INC., KITCHENER (CA)

(72) Inventors: ANDRE NOVOMIR HLADIO, WATERLOO (CA); JOSEPH ARTHUR SCHIPPER, KITCHENER (CA)

(21) Appl. No.: 17/610,865

(22) PCT Filed: May 14, 2020
(86) PCT No.: PCT/CA2020/050650

§ 371 Date: Nov. 12, 2021

Related U.S. Application Data
(60) Provisional application No. 62/848,058, filed on May 15, 2019.

(51) Int. Cl. A61B 5/107; A61B 34/10; G16H 20/40

(52) U.S. Cl. CPC A61B 5/1071; G16H 20/40; A61B 34/10; A61B 5/1072

(57) ABSTRACT

Systems, apparatus and methods are described to measure femoral version or other femoral information to provide clinically relevant information for total or partial hip arthroplasty. Anatomical measurements such as a femoral (shaft) axis and native femoral version may be provided from a planning system to a navigational system configured to track relative positions of a femur and pelvis during a procedure. A tracking element location may be defined (in the planning system or the surgical navigation system such as from 3D surface information received from the planning system). An invariable relationship between the tracking element location and the femoral axis may be leveraged to measure femoral information including post-procedure.

### FIELD

The present application relates to computer processing to measure objects such as parts of a human body and a surgical tool or implant in a 3D space and more particularly to systems and methods for computer assisted surgery to providing femoral version and/or other femoral or leg feature tracking.

### BACKGROUND

Hip replacements are common orthopedic surgeries wherein at least a portion of a patient's hip joint is replaced with an implant. As shown in FIG. 1 (Source: commons.wikimedia.org/wiki/File:903_Multiaxial_Joint.jpg), the hip joint 100 comprises two main components—the acetabulum 102 and the femoral head 104. A total hip arthroplasty (THA) is a procedure in which a surgeon will replace both the acetabulum 102 and the femoral head 104 with appropriately sized implants. A hemiarthroplasty is a procedure in which the patient's femoral head 104, but not the acetabulum 102, will be replaced with a femoral implant.

One clinically relevant parameter in hip arthroplasty (a reconstruction procedure) comprises femoral version as normal femoral version contributes to hip stability. Correcting for abnormal femoral version or maintaining existing normal femoral version may assist to enhance patient outcomes in a reconstruction procedure such as hip arthroplasty. Other femoral or leg features such as offset, leg length, etc. are also clinically relevant.

### SUMMARY

Systems, apparatus and methods are described to measure femoral version or other femoral information to provide clinically relevant information for total or partial hip arthroplasty. Anatomical measurements such as a femoral (shaft) axis and native femoral version may be provided from a planning system to a navigational system configured to track relative positions of a femur and pelvis during a procedure. A tracking element location may be defined (in the planning system or the surgical navigation system such as from 3D surface information received from the planning system). An invariable relationship between the tracking element location and the femoral axis may be leveraged to measure femoral information including post-procedure.

There is provided a system for measuring femoral information of a patient during a hip surgery, the system comprising: a localization system for measuring relative pose between a reference element on a pelvis and a tracking element on a femur of the patient, the tracking element positioned at a landing pad location (LPL), wherein: the LPL has an invariable location relationship to a first item of femoral information comprising or determinable from an anatomical measurement of the femur determined prior to the hip surgery, the first item of femoral information is useful to determine a second item of femoral information representing a clinically relevant measure changeable by the hip surgery; and the LPL and anatomical measurement are defined using femoral image space coordinates; and a computer storage device storing instructions, which when executed by a processor of a computing device, configure the computing device to: receive localization data where the tracking element is mounted at the LPL, the localization data defined using localization space coordinates; register the femur using the localization data to define a relationship between the femur navigation space coordinates and the localization space coordinates and to define a relationship between the femur navigation space coordinates and the femur image space coordinates; determine further femur anatomical information using localization data received, the further femur anatomical information defined using femur navigation space coordinates and the further femur anatomical information useful to determine the second item of femoral information; determine and present the second item of femoral information using: LPL; the anatomical measurement; the further femur anatomical information; and a relationship between the femur navigation space coordinates and the femoral image space coordinates.

The following are features and each many be combined with another unless indicated to the contrary.

The system may comprise the computing device. The storage device may be a component of the computing device.

The second item may comprise one of: femoral version, femoral offset; leg length change; anteroposterior leg change; and global offset.

The anatomical measurement may comprise the femoral axis or information to determine the femoral axis.

The instructions, when executed, may configure the computing device to verify that the tracking element is properly located at the LPL.

The system instructions, when executed, configure the computing device to use LPL and BPTE to provide information to register the femur. The femur may be registered in accordance with various manners. The instructions, when executed, may configure the computing device to register the femur in response to a holding of the femur in a neutral position relative to a registered pelvis. The instructions, when executed, may configure the computing device to use, further femoral anatomical information comprising BHCOR and, optionally, pre-operatively determined anatomical measurements to provide information to register the femur. The instructions, when executed, may configure the computing device to register the femur in response to input generated from a probing of landmarks on the femur. The instructions, when executed, may configure the computing device to register the femur by performing a surface mapping to a 3D model of the boney surface.

The instructions, when executed, may configure the computing device to receive in a data transfer from a planning system: the anatomical measurement; and one of: the LPL; and 3D surface information in femur image space coordinates with which to define the LPL. The data transfer may comprise one of a QR code data transfer where a QR code encodes the data to be received and a communication network transfer where the data is communicated via a network with a resource outside an OR. The 3D surface information comprises one of: a subset of a segmented CT scan; a mesh of nodes or a point cloud; a plurality of coefficients of a generic model; and a look up code for a library of shapes residing in memory on the computer. The instructions, when executed, may configure the computing device to use the 3D surface information to present a GUI displaying a representation of the femur and to receive input to define the LPL.

To receive input to define the LPL may comprise one of: receiving input in response to a selecting a point on a 3D representation determined from the 3D surface information; and receiving input in response to an aligning of cross hairs in two orthogonal views representing the femur from the 3D surface information.

To define the LPL may comprise performing calculations to determine optimal locations on the 3D surface for the LPL. The system of claim 15 wherein optimal means one or both of: being easily identifiable; and insensitive to positioning errors.

The LPL may comprise one of: a single point; and a region defined from multiple points.

The instructions, when executed, may configure the computing device to confirm an identity of the patient.

There is provided a method comprising: receiving localization data from a localization system for measuring relative poses between a reference element on a pelvis and a tracking element on a femur of a patient during a hip surgery, the tracking element positioned at a landing pad location (LPL) and wherein: the LPL has an invariable location relationship to a first item of femoral information comprising or determinable from an anatomical measurement of the femur determined prior to the hip surgery; and the first item of femoral information is useful to determine a second item of femoral information representing a clinically relevant measure changeable by the hip surgery; determining further femur anatomical information using localization data received, the further femur anatomical information useful to determine the second item of femoral information; and determining and presenting the second item of femoral information using: LPL; the anatomical measurement; and the further femur anatomical information.

It will be understood that for each of the system features there is provided a comparable method feature.

There is provided a tangible non-transitory computer readable medium storing instructions which when executed by a computing device configure the computing device to perform any of the method aspects.

There is provided a computing device for measuring femoral information of a patient during a hip surgery, the computing device comprising a memory device storing instructions which when executed by a processor of the computing device configure the computing device to perform any of the method aspects.

These and other aspects will be apparent to a person of ordinary skill in the art.

### BRIEF DESCRIPTION OF THE DRAWINGS

FIG. 1 is an illustration of a hip joint.

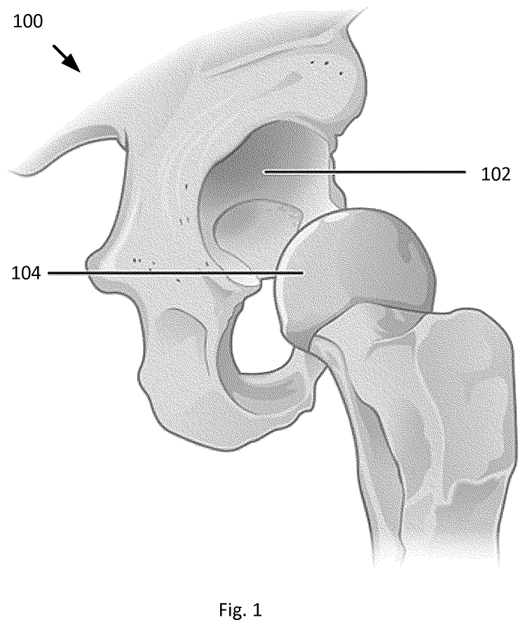

FIGS. 2A, 2B and 2C are illustrations of normal femoral version, femoral anteversion and femoral retroversion.

FIGS. 3A and 3B are flowcharts for a planning system and surgical navigation system in accordance with respective examples.

FIG. 4 is an illustration of a femur portion of FIG. 2A showing anatomical measurements and 3D surface information that comprise data that may be transferred from the planning system in accordance with the examples of FIGS. 3A and 3B.

FIG. 5 is an illustration of a combined computing system showing a planning system and a surgical navigation system where the surgical navigation system is shown during a procedure, in accordance with an example.

FIG. 6 is an illustration of a graphical user interface of surgical navigation system of FIG. 5 in accordance with an example.

The present inventive concept is best described through certain embodiments thereof, which are described herein with reference to the accompanying drawings, wherein like reference numerals refer to like features throughout. It is to be understood that the term invention, when used herein, is intended to connote the inventive concept underlying the embodiments described below and not merely the embodiments themselves. It is to be understood further that the general inventive concept is not limited to the illustrative embodiments described below and the following descriptions should be read in such light. More than one inventive concept may be shown and described and each may standalone or be combined with one or more others unless stated otherwise.

### DETAILED DESCRIPTION

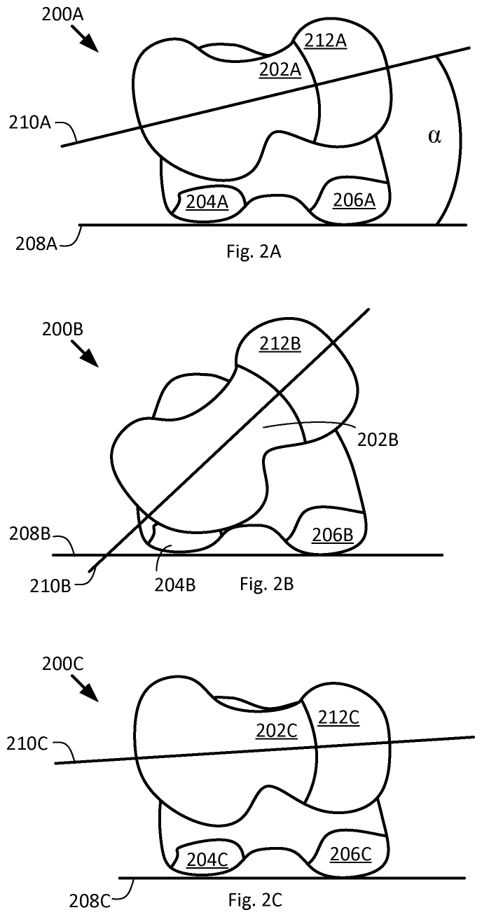

Reference is directed to FIGS. 2A, 2B and 2C depicting illustrations of normal femoral version, femoral anteversion and femoral retroversion respectively for femurs 200A, 200B and 200C. Femoral version means the orientation of the femoral neck 202A, 202B and 202C in relation to the femoral condyles (respectively 204A, 206A; 204B, 206B; and 204C, 206C) providing a respective transcondylar axis (e.g. 208A, 208B and 208C) at the knee (only a femoral component of a knee is shown). Transcondylar axis may be a plane.

A femoral neck axis (e.g. 210A, 210B and 210C) extends through the respective femoral neck 202A, 202B and 202C and a respective femoral head (e.g. 212A, 201B and 212C). For purposes of illustration neck axis is shown on the surface. The femoral neck axis may be a plane. Normal femoral version (FIG. 2A) has a relative angle (α) of 15°-20° between transcondylar axis 208A and the femoral neck axis 210A.

Femoral anteversion (FIG. 2B) refers to an example where the femoral neck axis 210B is rotated anterior with respect to the transcondylar axis 208B, with the femoral head 212B directed anterior to the coronal plane of the femur 200B. In contrast, femoral retroversion (FIG. 2C) refers to the femoral neck axis 210C being oriented posterior to the transcondylar axis 208C such that the femoral neck 202C and femoral head 212C is positioned posterior to the coronal plane of the femur 210C.

As femoral version is a clinically relevant measure desired for use during at least some reconstruction procedures, described are systems, methods and apparatus for measuring femoral version information. Femoral version information may be measured during a procedure such as to measure native femoral version information (e.g. prior to removal of a portion of the femur), or following an implanting of a trial implant or following implanting of a final implant to provide reconstructed femoral version information. Any of the measured femoral version information may be compared to each other. Any of the measured femoral version information may be compared to independently determine native femoral version information. Independently determined native femoral version information is determined in an alternate manner than measuring by a surgical navigation system such as by using a planning system. Any of the measured femoral version information may be compared to target femoral version information representing a desired position (which too may be determined using a planning system).

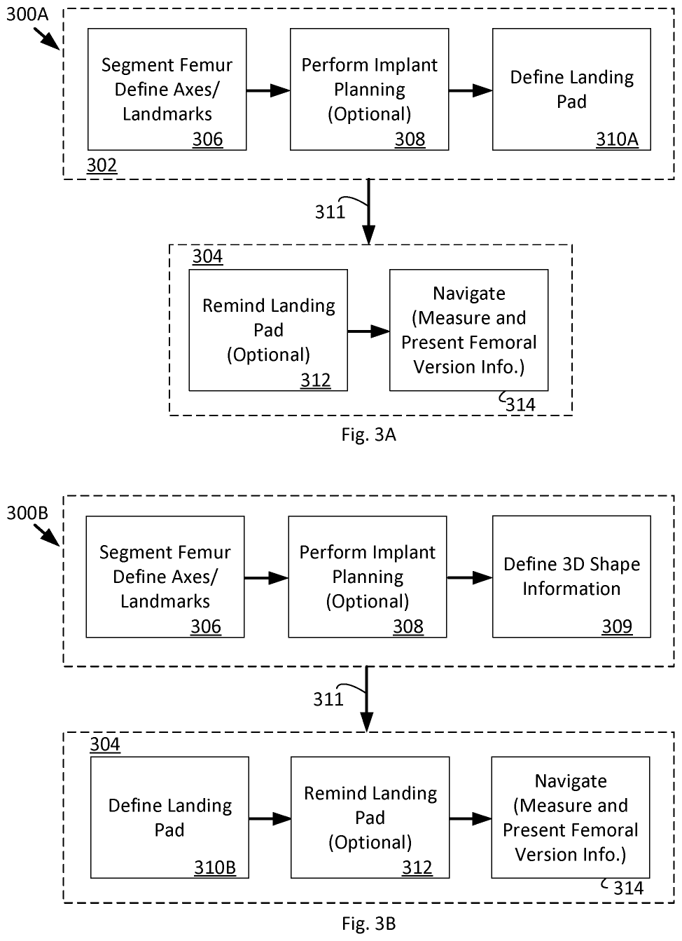

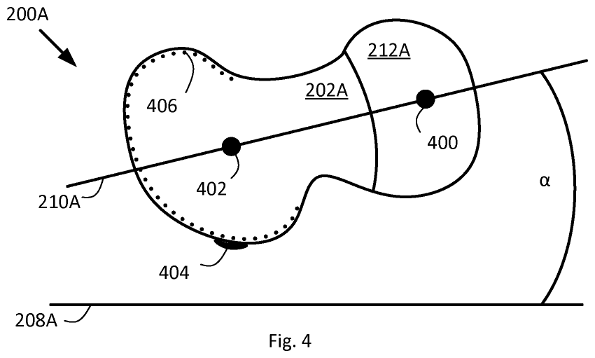

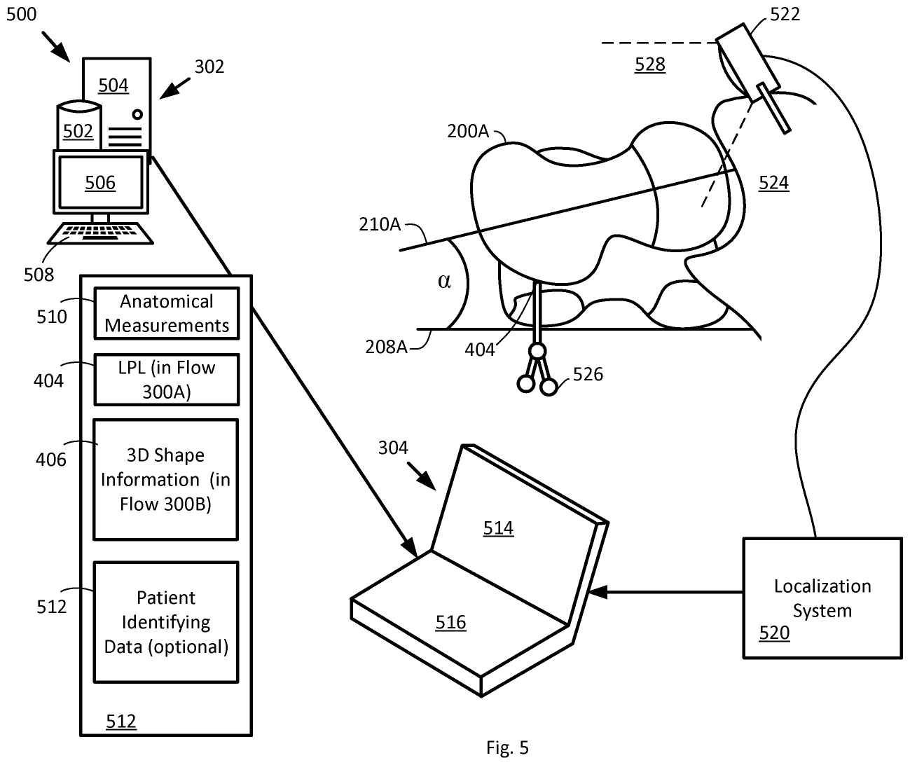

Independently determined native femoral version information as well as other native anatomical measurements for a patient, particularly the patient femur may be determined and made available from a planning system to a surgical navigation system such as for use during the reconstruction procedure. FIGS. 3A and 3B show two process flows 300A and 300B to define, transfer and use information, particularly anatomical measurements, in relation to operations of a planning system 302 and a surgical navigation system 304. FIG. 4 is an illustration of a portion of a femur of FIG. 2A showing anatomical measurements. FIG. 5 is an illustration of a combined computing system 500 comprising planning system 302 and surgical navigation system 304 in accordance with an embodiment. It is clear that the components shown in FIG. 5 are not to scale.

The two process flows 300A and 300B relate to alternatives. In flow 300A, planning system 302 is shown having operations 306, 208 and 310A while surgical navigation system 304 is shown having operations 312 and 314. In flow 300A, arrow 311 represents communications between operations 310A and 312 representing a data transfer between planning system 302 and surgical navigation system 304. In flow 300B, planning system 302 is shown having operations 306 and 308 while the surgical navigation system 304 is shown having operations 310B, 312 and 314. Data transfer operations 311 are shown between operations 308 and 310B in flow 300B.

Planning system 302 may receive image data for a patient such as a computerized tomography (CT) scan in relation to the femur (and typically also the pelvis, etc.). The image data may be stored in a data base 502 coupled to or part of planning system 302. Planning system 302 comprises a computing device 504 having or coupled to a display 506 and input device(s) such as a keyboard 508. Other input devices may include a microphone, pointing device, touchscreen, etc. (not shown). Other output devices not shown may include a speaker, lights, etc. Planning system 302 may be configured such as via instructions (software) stored in a storage device (e.g. a memory or other non-transitory medium) for execution by a processor. The instructions may provide a graphical user interface to present image data and receive input such as to define anatomical measurements relative to the image data. Planning system 302 may be configured via workflow (from the software instructions) to guide a user to provide input for the operations of planning system 302.

At 306, operations generate a 3D model of the femur (e.g. via segmentation of a CT-scan) and define anatomical measurements 510 comprising femoral landmarks and axes, for example, responsive to user input, for example, from a surgeon (not shown). Anatomical measurements 510 are defined in a coordinate space for the 3D model of the femur, referred to herein as a femur image coordinate space. To assist with illustrating the anatomical landmarks and other information, FIG. 4 shows a portion of the femur 200A of FIG. 2A with the distal portion of the femur not shown for clarity. With reference to FIGS. 2A and 4 anatomical measurements 510 may comprise a baseline hip centre of rotation (BHCOR) 400. It is understood that a BHCOR is a position within the femoral head 212A though in the present view it appears upon a surface thereof for purposes of illustration.

Anatomical measurements 510 may comprise landmarks for distal condyls (e.g. 204a and 206A) and/or the transcondylar axis (e.g. 208A). The transcondylar axis may be determined from the landmarks for the distal condyls. Anatomical measurements 510 may comprise a central femoral axis 402 running longitudinally along the femur. It is understood that femoral axis 402 is a position within the femur though in the present view it appears upon a surface thereof for purposes of illustration.

Anatomical measurements 510 may comprise a femoral head axis (e.g. 210A). It is also understood that the femoral head axis 210A may be determined from the BHCOR position and the femoral axis as a line or plane passing through (intersecting) the two. Anatomical measurements 510 may comprise native femoral version information such as angle α as previously described. It is understood that angle α may be determined from anatomical measurements such as the femoral head axis and the transcondylar axis. As noted, an axis may be a plane.

At 308 operations of planning system 302 may perform implant planning responsive to input from a user. For example, a target femoral version (e.g. a desired value for angle α) optionally may be defined (e.g. as an anatomical measurement 510 or otherwise such as a numerical value). For the purposes of the present disclosure, such planning steps per se are optional.

At 310, operations responsive to user input define a landing pad location (LPL) 404 to define a target location for placement of a surgical navigation tracker element. The LPL 404 may be defined as a point or a region (e.g. a plurality of points defining a shape) on a surface of the femur. Like the anatomical measurements, the LPL is defined in the femur image coordinate space.

As will be described in greater detail below, LPL 404 is useful to determine measured femoral version information. In brief, an invariable relationship between the location of the LPL and a second location of an anatomical feature of the femur that is relevant to determining femoral version and which second location does not change following a reconstruction procedure may be used to measure femoral version using surgical navigation techniques. The second location may be a position along the central femoral axis 402. The planning system 302 provides to the surgical navigation system 304 the anatomical measurements and LPL where the anatomical measurements comprise the BHCOR and either the second location comprising the femoral axis (FA) or a native femoral version (e.g. angle α) and a distance measure from the BHCOR to the FA. The surgical navigation system may determine mapping between its coordinate space and the coordinate space of the planning system and use the information to measure femoral version. As the LPL on the femur does not change as a result of the surgical procedure and as the femoral axis also does not change, the relationship between the LPL and central axis is fixed throughout. The surgical navigation system 304 can verify the location of the LPL in a localization coordinate space and its own femur navigation space (e.g. before and after surgery). With this information, the surgical navigation space may use the relationship to the femoral axis to determine its location. The surgical navigation system 304 can also determine HCOR (e.g. post-surgery) and from this determine femoral version. The femoral version may be a delta (e.g. a difference measure) from a pre-surgery femoral version measure. For example, the delta may be +1° to be added to the native femoral version (e.g. angle α provided by the planning system 302).

It will be understood that if the surgical navigation system received the BHCOR and femoral axis anatomical measurements, it could determine the native femoral version. For example, some assumptions (or constraints) may be made about registration of the anatomy to the surgical navigation system. For example, it may be assumed that the BHCOR and femoral axis are determined in the planning software in a relevant coordinate system i.e. with respect to the transcondylar axis, and that the patient's leg is in a neutral position during registration. In this way, the transcondylar axis is along the medial-lateral pelvic direction permitting a determination of angle α. If the surgical navigation system receives anatomical measurements comprising BHCOR and native femoral version, information for such a version may be constrained to comprise an angle and a distance (not just an angle) to be able to determine the femoral axis value. Similar registration assumptions would be made as above.

At 311, operations transfer data 512 comprising the anatomical measurements and LPL to surgical navigation system 304. Data transfer may be an electronic communication between planning system 302 and surgical navigation system 304 and the two systems may be communicatively coupled accordingly. They are typically coupled in an indirect manner (not shown). An indirect manner may be a communication via one or more networks (not shown) to an intermediary computing device (not shown) and then a communication to surgical navigation system 304. It is understood that surgical navigation system 304 is located in an operating room (OR) when in use for a surgical procedure. An intermediary computing device may be located remotely outside the OR or locally inside the OR. An intermediary computing device may comprise a mobile computing device such as a smartphone, laptop, tablet, etc. or another form factor. One indirect manner of data transfer may comprise storing the data electronically on a portable storage device (e.g. CD ROM, USB drive, etc.) that is then coupled to surgical navigation system 304.

In another data transfer example, the data 512 may be encoded such as in a quick response (QR) code or other matrix barcode. Such may be stored electronically and/or printed and presented to optically transfer the information. In addition to anatomical measurements 510 and LPL 404, patient identifying data 512 may be transferred. Such data is typically available to planning system 302, for example, associated with planning data and/or image data. The surgical navigation system may be configured to confirm patient identity, for example, as a portion of workflow, to proceed with a procedure. Patient identity information may be presented and confirmed against patient identity information associated with the patient in an alternate manner, for example, in records etc. present in an OR with the patient. In this manner, surgical navigation system may be confirmed to be using correct anatomical measurements, etc. received for the patient.

Surgical navigation system 304 comprises a computing device (e.g. a laptop, PC or other form factor) having or coupled to a display 514 and input device(s) such as a keyboard 516. Other input devices may include a microphone, pointing device, touchscreen, etc. (not shown). Other output devices not shown may include a speaker, lights, etc. Surgical navigation system 304 may be configured such as via instructions (software) stored in a storage device (e.g. a memory or other non-transitory medium) for execution by a processor. The instructions may provide a graphical user interface to present localization data, image data and workflow for assisting with a surgical procedure such as a reconstruction procedure for a patient's hip.

Surgical navigation system 304 may be configured via workflow (from the software instructions) to guide a user to receive data from planning system 302 and configure (e.g. set up) a localization system 520 to enable the surgical navigation system 304 to receive localization data to determine relative measurements of patient anatomy, surgical tools or other objects tracked by the localization system 520. Localization system 520 is coupled to receive sensor data from a camera 522 (e.g. a reference element) coupled to a pelvis 524 (shown partially cut away) for a tracking element 526 located in a field of view 528 of the camera 522. The tracking element is coupled to femur 200A at LPL 404.

At 312, operations optionally remind the user of the LPL. A reminder may display the LPL on an image for example. The LPL may be visualized on a femur such as by displaying images of the femur (e.g. of the actual patient femur or representational of a typical femur) in two planes such as two orthogonal planes to guide the placement in 3D, or by displaying the LPL on a 3D representation of the patient femur.

At 314, navigation operations are performed, for example, to measure and present femoral version information. It is understood that a registration operation is undertaken. The localization system presents sensor data in localization space coordinates. The planning system provides anatomical measurements (and LPL) in femur image space coordinates. The surgical navigation system utilizes a femur navigation space. These various spaces are registered to determine transformations (mathematical operations or a map) so the respective data from each may be used together.

FIG. 3B shows flow 300B. The operations therein are similar to those of flow 300A. In flow 300B rather than define the LPL using the planning system 302, the LPL is defined using the surgical navigation system 304. Planning system 302 (or in another manner) may provide 3D surface information 406 (represented by the line of dots for clarity about a portion of the surface of femur 200A. It is understood that the 3D surface information is not a single line or contour per se but representative of at least a region of the 3D surface of the femur. While planning system 302 could provide a fulsome 3D model or even the raw data (e.g. the CT-scan from which a fulsome 3D model may be defined) as the 3D surface information, it may be sufficient to transfer a reduced amount of data, for example, which may define 3D information for a region (a portion of interest about the LPL for example) of the surface of the femur or less detailed information about a greater portion or even all of the femur.

Where the 3D surface information is reduced data (e.g. less than the 3D model of the planning system and not the raw data), the 3D surface information 406 may comprise a subset of a segmented CT scan. It may comprise a mesh of nodes or a point cloud. It may comprise coefficients of a generic (3D) model of the femur. It may comprise a look up code or plurality of codes for a library of shapes residing in a storage device available to surgical navigation system 304.

Planning system 302 may be configured to define this 3D surface information 406 (at step 309). At 311 data 512 is communicated. FIG. 5 shows data 512 for both flow options such that LPL 404 is transferred for flow 300A and 3D surface information 406 is transferred for flow 300B. Patient identifying data is optionally transferred.

At 310B the LPL 404 is defined from the 3D shape data in response to input received from a user.

To receive input to define the LPL based on 3D surface information may comprise receiving input in response to a selecting of a point on a 3D visualization of the 3D surface information such as in a GUI of system 302 (not shown).

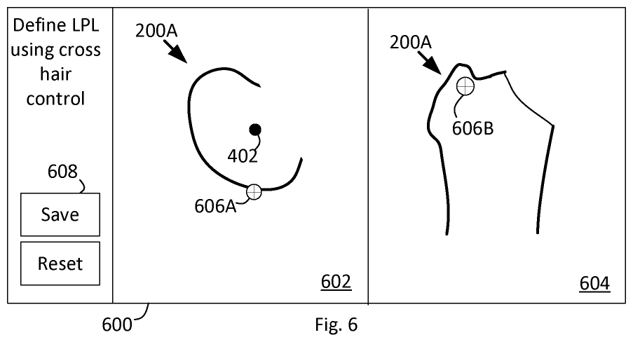

In an example shown in FIG. 6, a GUI 600 (a screenshot illustration) may present two orthogonal views of the femur 200A derive from the 3D surface information 406 in portions 602 and 604 of the GUI 600. The 3D surface information may provide only a portion of the femur and thus views in 602 and 604 are portions thereof. GUI 600 may provide a control 606A, 606B comprising cross hairs (an aiming device) in each of the portions that may be manipulated via input to move the controls in both portions relative to the views to select the LPL on a point or region of the 3D surface and save (e.g. via control 608). A similar GUI (not shown) may be configured for planning system 302 with which to define the LPL 404 for surgical navigation system 304 such as in a 3D mode or in 2 or 3 orthogonal views (e.g. CT slices).

In an example, to define the LPL (in either the planning system 302 or the surgical navigation system 304) may comprise performing calculations to determine more/less optimal locations on the 3D surface for the LPL. Optimal means one or both of: being easily identifiable; and insensitive to positioning errors. To be “identifiable”: a parameter indicating accessibility within the surgical wound may be generated. A priori information may be leveraged, such as the surgical approach (e.g. for an anterior approach hip replacement, the anterior aspect of the greater trochanter is accessible; a parameter associated with “how anterior” the LPL is may be generated and used to determine more/less optimal positions. Proximity to standard anatomical landmarks may be a parameter of “identifiability”. They may allow a surgeon to place the actual tracking element more accurately with respect to the LPL since they can place it in reference to an identifiable anatomical feature.

To be insensitive: a parameter based on the gradient of the femur 3D model (or 3D surface information) as well as the anatomical information may be used to indicate regions where positioning errors (i.e. where LPL and tracking element locations do not match) have minimal effect on calculating femur version information. This can be done using optimization techniques/sensitivity analysis.

In both cases, there may be displayed visually this information for the user during LPL selection (whether done in the planning system 302 or the navigation system 304).

Performing such calculations may be performed by using a user-identified region for possible locations, and performing perturbation simulations which measure the change in femoral version measurements with error in LPL placement using the 3D surface information. These simulations may be specific to the user selected targets, or may be simulated using a range of generic targets. These simulations would take into account likely magnitudes of placement error, and the femoral geometry of the defined area. Alternatively, the system may only calculate the sensitivity of a defined LPL, and report that to the user. The user could manually adjust the LPL in the planning software until the reported sensitivity is acceptable according to their preference.

It is understood that the LPL as received from the planning system 302 or defined using the 3D surface information by system 304 is in relation to the femur image coordinate system.

During the reconstruction procedure (e.g. hip surgery) the localization system measures relative pose(s) between the reference element 522 on the pelvis 524 and a tracking element 526 on the femur. It will be understood that the user mounts the tracking element at the location indicated by the LPL. Mounting maybe directly to the femur or indirectly via a device. The location of the device may be probed (i.e. a probe used to indicate the location to the navigation system 304).

The compute device has access to (e.g. because it has received such via a data transfer) anatomical measurements comprising a baseline hip center of rotation (BHCOR) and native anteversion information and/or femoral axis information in femur image space coordinates as well as LPL. The LPL may have been received from the planning system or may be determined by the surgical navigation system 304 using 3D surface information 406 for the femur from the planning system 304.

Localization data is received to determine the position of the tracking element in femur navigation space coordinates. Registration is performed and described further below.

The computing device may calculate a baseline hip center of rotation (BHCOR) in localization space coordinates using data from the localization system. For example, the leg is rotated in the hip joint and multiple measurements taken to determine the COR.

A baseline position of the tracking element (BPTE) is determined in localization space coordinates using localization data received.

Following reconstruction, an implant hip center of rotation (IHCOR) is calculated in localization space coordinates using localization data received. Again, the leg may be rotated and localization measurements taken.

Following reconstruction, a femoral implant replaces the original femoral head (and possibly the acetabulum is amended or lined with a (new) cup. A reconstructed position of the tracking element (RPTE) is determined in localization space coordinates using localization data.

Femoral version information may be calculated based on BHCOR, IHCOR, BPTE, RPTE, anatomical measurements and LPL.

The femoral axis (FA) and the LPL may be defined during planning in the femur image space coordinates.

The system may register the femur using the LPL, and BPTE, by defining their positions to be coincident, and by defining the orientation of the femur navigation space coordinates and femur image space coordinates to be aligned with pelvic coordinates while aligning the leg in a neutral position during this step.

Registering the femur is understood to mean defining the spatial relationship(s) between the localization space coordinates, the femur navigation space coordinates, and the femur image space coordinates.

The BHCOR and IHCOR can be represented in the femur navigation space coordinates using the BPTE, and the RPTE respectively, and the relationship of the tracking element to the femur navigation space coordinates.

The baseline femoral version (BFV) can be calculated as the angle of the vector from the BHCOR to the FA (in femur navigation space coordinates) projected onto the transverse axis. The reconstructed femoral version (RFV) can be calculated in a similar manner using the IHCOR and the FA. A change in femoral version can be calculated as the difference between the BFV and the RFB.

The FA may alternatively be defined with a BHCOR measured in femur image space coordinates, BFV, and a distance between the BHCOR and the FA. The femur navigation space coordinates may be defined in relation to the BPTE using intra-operative measurements of landmarks, such as the patella, the greater trochanter, and the BHCOR.

The system may also define the femur navigation space coordinates by registering probed points in localization space coordinates to 3D model information of the femur.

The system may use LPL, BHCOR and BPTE, and optionally pre-operatively determined anatomical measures to verify that the tracking element is properly located. If the hip center is measured during planning relative to the LPL, than the relative positions of the two points should be the same when measured during surgery (the BHCOR and BPTE). Any discrepancy between the measurements could be used to identify misplacement of the BPTE, (relative to the expected LPL), or be used to correct errors.

Exemplary methods and system for registering and/or tracking the location of surgical tools and patient anatomy are described in applicant's U.S. Pat. Nos. 9,713,506 and 9,603,671, the contents of which are hereby incorporated herein by reference in their entirety.

In addition to femoral version, the surgical navigation system may determine other items of femoral information. The surgical navigation system 304 may use the same anatomical measurements and localization data calculate other clinically relevant parameters. One such parameter may be femoral offset, defined as the length of the vector from the hip center of rotation to the femoral axis, when projected on the transverse plane. A change in femoral offset could be calculated from a femoral offset measured at a baseline measurement position, and after the joint has been reconstructed.

The same anatomical measurements together with localization data may be used to measure other clinically relevant parameters such as leg length change, anteroposterior leg change, global offset, etc. These parameters have well established clinical definitions, and one skilled in the art could understand how to calculate these and other parameters using the available information.

It is also understood that the surgical navigational system may be communicatively coupled to a robot for performing aspects of the reconstruction surgery.

In addition to system, apparatus and method aspects, there is provided a tangible non-transitory computer readable medium storing instructions which when executed by a computing device configure the computing device to perform any of the methods as described.

Practical implementation may include any or all of the features described herein. These and other aspects, features and various combinations may be expressed as methods, apparatus, systems, means for performing functions, program products, and in other ways, combining the features described herein. A number of embodiments have been described. Nevertheless, it will be understood that various modifications can be made without departing from the spirit and scope of the processes and techniques described herein. In addition, other steps can be provided, or steps can be eliminated, from the described process, and other components can be added to, or removed from, the described systems. Accordingly, other embodiments are within the scope of the following claims.

Throughout the description and claims of this specification, the word “comprise” and “contain” and variations of them mean “including but not limited to” and they are not intended to (and do not) exclude other components, integers or steps. Throughout this specification, the singular encompasses the plural unless the context requires otherwise. In particular, where the indefinite article is used, the specification is to be understood as contemplating plurality as well as singularity, unless the context requires otherwise. By way of example and without limitation, references to a computing device comprising a processor and/or a storage device includes a computing device having multiple processors and/or multiple storage devices. Herein, “A and/or B” means A or B or both A and B.

Features, integers characteristics, compounds, chemical moieties or groups described in conjunction with a particular aspect, embodiment or example of the invention are to be understood to be applicable to any other aspect, embodiment or example unless incompatible therewith. All of the features disclosed herein (including any accompanying claims, abstract and drawings), and/or all of the steps of any method or process so disclosed, may be combined in any combination, except combinations where at least some of such features and/or steps are mutually exclusive. The invention is not restricted to the details of any foregoing examples or embodiments. The invention extends to any novel one, or any novel combination, of the features disclosed in this specification (including any accompanying claims, abstract and drawings) or to any novel one, or any novel combination, of the steps of any method or process disclosed.

1. A system for measuring femoral information of a patient during a hip surgery, the system comprising:
a localization system for measuring relative pose between a reference element on a pelvis and a tracking element on a femur of the patient, the tracking element positioned at a landing pad location (LPL), wherein:
the LPL has an invariable location relationship to a first item of femoral information comprising or determinable from an anatomical measurement of the femur determined prior to the hip surgery,
the first item of femoral information is useful to determine a second item of femoral information representing a clinically relevant measure changeable by the hip surgery; and
the LPL and anatomical measurement are defined using femoral image space coordinates; and
a computer storage device storing instructions, which when executed by a processor of a computing device, configure the computing device to:
receive localization data where the tracking element is mounted at the LPL, the localization data defined using localization space coordinates;
register the femur using the localization data to define a relationship between the femur navigation space coordinates and the localization space coordinates and to define a relationship between the femur navigation space coordinates and the femur image space coordinates;
determine further femur anatomical information using localization data received, the further femur anatomical information defined using femur navigation space coordinates and the further femur anatomical information useful to determine the second item of femoral information; and
determine and present the second item of femoral information using:
LPL;
the anatomical measurement;
the further femur anatomical information; and
a relationship between the femur navigation space coordinates and the femoral image space coordinates.

2. The system of claim 1 wherein the second item comprises one of: femoral version, femoral offset; leg length change; anteroposterior leg change; and global offset.

3. The system of claim 1 wherein the anatomical measurement comprises the femoral axis or information to determine the femoral axis.

4. The system of claim 1 wherein the instructions, when executed, configure the computing device to verify that the tracking element is properly located at the LPL.

5. The system of claim 1 wherein the instructions, when executed, configure the computing device to use LPL and BPTE to provide information to register the femur.

6. The system of claim 5 wherein the instructions, when executed, configure the computing device to register the femur in response to a holding of the femur in a neutral position relative to a registered pelvis.

7. The system of claim 5 wherein the instructions, when executed, configure the computing device to use, further femoral anatomical information comprising BHCOR and, optionally, pre-operatively determined anatomical measurements to provide information to register the femur.

8. The system of claim 5 wherein the instructions, when executed, configure the computing device to register the femur in response to input generated from a probing of landmarks on the femur.

9. The system of claim 5 wherein the instructions, when executed, configure the computing device to register the femur by performing a surface mapping to a 3D model of the boney surface.

10. The system of claim 1 wherein the instructions, when executed, configure the computing device to receive in a data transfer from a planning system:
the anatomical measurement; and
one of:
the LPL; and
3D surface information in femur image space coordinates with which to define the LPL.

11. The system of claim 10 wherein the data transfer comprises a:
a QR code data transfer where a QR code encodes the data to be received; and
a communication network transfer where the data is communicated via a network with a resource outside an OR.

12. The system of claim 10 wherein the 3D surface information comprises one of:
a subset of a segmented CT scan;
a mesh of nodes or a point cloud;
a plurality of coefficients of a generic model; and
a look up code for a library of shapes residing in memory on the computer.

13. The system of claim 10 wherein the instructions, when executed, configure the computing device to use the 3D surface information to present a GUI displaying a representation of the femur and to receive input to define the LPL.

14. The system of claim 13 wherein to receive input to define the LPL comprises one of:
receiving input in response to a selecting a point on a 3D representation determined from the 3D surface information; and
receiving input in response to an aligning of cross hairs in two orthogonal views representing the femur from the 3D surface information.

15. The system of claim 10 wherein to define the LPL comprises performing calculations to determine optimal locations on the 3D surface for the LPL.

16. The system of claim 15 wherein optimal means one or both of:
being easily identifiable; and
insensitive to positioning errors.

17. The system of claim 1 wherein the LPL comprises one of:
a single point; and
a region defined from multiple points.

18. The system of claim 1 wherein the instructions, when executed, configure the computing device to confirm an identity of the patient.

19. A method comprising:
receiving localization data from a localization system for measuring relative poses between a reference element on a pelvis and a tracking element on a femur of a patient during a hip surgery, the tracking element positioned at a landing pad location (LPL) and wherein:
the LPL has an invariable location relationship to a first item of femoral information comprising or determinable from an anatomical measurement of the femur determined prior to the hip surgery; and
the first item of femoral information is useful to determine a second item of femoral information representing a clinically relevant measure changeable by the hip surgery;
determining further femur anatomical information using localization data received, the further femur anatomical information useful to determine the second item of femoral information; and
determining and presenting the second item of femoral information using:
LPL;
the anatomical measurement; and
the further femur anatomical information.

20. A tangible non-transitory computer readable medium storing instructions which when executed by a computing device configure the computing device to perform a method comprising:
receiving localization data from a localization system for measuring relative poses between a reference element on a pelvis and a tracking element on a femur of a patient during a hip surgery, the tracking element positioned at a landing pad location (LPL) and wherein:
the LPL has an invariable location relationship to a first item of femoral information comprising or determinable from an anatomical measurement of the femur determined prior to the hip surgery; and
the first item of femoral information is useful to determine a second item of femoral information representing a clinically relevant measure changeable by the hip surgery;
determining further femur anatomical information using localization data received, the further femur anatomical information useful to determine the second item of femoral information; and
determining and presenting the second item of femoral information using:
LPL;
the anatomical measurement; and
the further femur anatomical information.

21. (canceled)
